(19) United States
(12) Patent Application Publication
(10) Pub. No.: US 2005/0158734 A1
(43) Pub. Date: Jul. 21, 2005

(54) Alleles corresponding to various diet-associated phenotypes

(76) Inventor: James Kaput, Justice, IL (US)

Correspondence Address: BELL & ASSOCIATES, 416 FUNSTON ST., SUITE 100, SAN FRANCISCO, CA, 94118, US

(21) Appl. No.: 10/914,723

(22) Filed: Aug. 9, 2004

Related U.S. Application Data
(63) Continuation-in-part of application No. 10/700,305, filed on Oct. 31, 2003.
(60) Provisional application No. 60/423,104, filed on Nov. 1, 2002.

(51) Int. Cl. C12Q 1/68; C12M 1/34

(52) U.S. Cl. 435/6; 435/287.2; 600/300

(57) ABSTRACT

Diet-regulated disease-associated genes (whose regulation differed among various genotypes and diet combinations.

This application is a continuation in part of currently pending U.S. application Ser. No. 10/700,305 filed Oct. 31, 2003 which itself claims the benefit of U.S. provisional application No. 60/423,104 filed Nov. 1, 2002. Both of these applications are hereby incorporated by reference in their-entirety.

### FIELD OF THE INVENTION

The field of the invention encompasses diet-regulated disease-associated genes, methods for identifying such genes and genes identified by such methods. The invention also concerns methods of monitoring treatment efficacy and disease progression by measuring the change in expression of diet-regulated disease-associated genes.

### BACKGROUND

Diet plays a major role in gene expression and disease causation and progression. Altering the concentration of a single metabolite often has pleioropic effects on highly disparate areas of disease-related physiology.

Diabetes is a well-known example of a diet-regulated disease in which nutrient-regulated messengers (insulin and glucagon) play a key role. Obesity is another well-known diet related disease in which nutrient-regulated messengers such as leptins, adiponectin, and resistin are important control factors. These conditions, and many other chronic diseases, are caused by multiple genes influenced by many environmental factors. The complexity of these diseases makes them difficult to diagnose and treat. Specifically, three key factors may affect the diagnosis and treatment of chronic diseases:
a) The same disease phenotype may result from disturbance in different metabolic pathways
b) The genetic makeup of each human differs, causing variation in response to the same factors
c) Environmental factors, such as diet, influence health and disease development

Chronic diseases, including obesity, Alzheimer's, diabetes, cardiovascular diseases, and certain cancers (among others), are generally produced by the interplay of environmental factors and genetic mechanisms. In addition, different members of the population showing clinical symptoms of any given disease can be grouped with each group having some unique genes or ESTs that contribute to disease formation. Furthermore, subsets of the unique and commonly-distributed genes are regulated directly or indirectly by foods chemicals. Genes that are diet-regulated and involved in disease processes can be identified and grouped to provide diagnostic markers and targets for drugs.

### BRIEF DESCRIPTION OF THE INVENTION

Methods for identifying diet-regulated disease-associated polynucleotides have already been disclosed in the previously-filed, related patent application Ser. No. 10/700,305, incorporated by reference herein. The present disclosure additionally sets out a number of genes that have been newly-identified as being diet-regulated disease-associated genes. This identification has been done by employing the methods disclosed in the parent application and re-analyzing the data disclosed in the parent application. The present disclosure newly identifies 388 diet-regulated disease-associated genes (whose regulation differed among various genotypes and diet combinations. Of these 388 genes the functions of 223 are of known.

Briefly, the invention encompasses the use of the 388 newly-identified diet-regulated disease-associated genes in arrays, the use of such arrays to measure the expression of these genes in individuals and in populations, and the use of such screening procedures to provide data useful in the formulation of foods that are beneficial to individuals and populations.

A microarray for screening for the presence of variants (alleles) of one or more diet-regulated disease-associated genes, the microarray comprising at least one nucleotide variant selected from the group consisting of: the genes listed in Table 2

A microarray for screening for the presence of variants (alleles) of one or more diet-regulated disease-associated genes, the microarray comprising the genes listed in Table 2.

A method for determining, in an individual, an association between an allele of a diet-regulated disease-associated gene and a diet-associated phenotypic response, by performing the following steps: (1) determining which allele(s) are present in the individual, (2) feeding the individual a first diet, (3) determining the phenotype of the individual in response to the first diet, (4) feeding the individual a second diet, (5) determining the new phenotype of the individual and (6) determining an association between an allele of a diet-regulated disease-associated gene and a diet-associated phenotype.

A method for determining the variants (alleles) of diet-regulated disease-associated genes in an individual, the method comprising screening the genome of the individual using the microarray described above and comparing the genotype to individuals not expressing a given phenotype.

A method for determining the variants (alleles) of diet-regulated disease-associated genes in an individual in response to two different diets, the method comprising the steps of: screening the genome of the individual using the microarray described above, then feeding the individual a defined diet and re-screening the individual using phenotypic analyses before and after the new diet.

A method for determining the variants (alleles) of diet-regulated disease-associated genes in a population, the method comprising screening the genome of the population using the microarray described above and comparing the presence of variants to the variants in individuals without evidence of disease levels to a known standard.

A method for determining the variants (alleles) of diet-regulated disease-associated genes in a population in response to two different diets, the method comprising: dividing the population into two groups, feeding each group a different defined diet, screening the genome of each group using the microarray described above, and comparing the variants (alleles) and phenotypic responses between the two groups.

A microarray for screening for the variants (alleles) of one or more diet-regulated disease-associated genes, the mircoarray comprising at least one nucleotide selected from the group consisting of: the genes listed in Table 3.

A microarray for screening for the variants (alleles) of one or more diet-regulated disease-associated genes, the microarray comprising the genes listed in Table 3.

A method for determining the variants (alleles) of diet-regulated disease-associated genes in an individual, the method comprising screening the genome of the individual using the microarray described above and comparing the variants (alleles) and phenotype to individuals with differing phenotypic expressions.

A method for determining the variants (alleles) of diet-regulated disease-associated genes in an individual in response to two different diets, the method comprising screening the genome of the individual using the microarray described above, then feeding the individual a defined diet and comparing phenotypic responses before and after the change in diet.

A method for determining the variants (alleles) of diet-regulated disease-associated genes in a population, the method comprising screening the genome of the population using the microarray described above.

A method for determining the variants (alleles) of diet-regulated disease-associated genes in a population in response to two different diets, the method comprising: dividing the population into two groups, feeding each group a different defined diet, screening the genome of each group using the microarray described above, and comparing phenotypic responses and their association to genotype.

A method for formulating a food beneficial to an individual, wherein the individual when fed a defined diet has an beneficial physiological response associated with the presence of variants (alleles) of at least one diet-regulated disease-associated gene listed in table 2 the method comprising the steps of: screening the genome of the individual using the microarray described above, then feeding the individual a defined diet and re-screening the individual using physiological measurements. Food formulation may have increased or reduced amounts, then formulating a food with a reduced content of these food elements. “Food elements” means any naturally occurring chemical or component of food, or chemicals or components that are manufactured to mimic naturally occurring chemicals or components. To say that a formulated food has a “reduced content” of a food element means that it has a content of the food element(s) that is substantially lower than would be received in a typical diet. It does not mean that the food element is absent, merely reduced by substantial amount.

A method for formulating a food beneficial to an individual, wherein the individual when fed a defined diet has a physiological response associated with the presence of certain variants (alleles) of at least one diet-regulated disease-associated gene listed in

A method for formulating a food beneficial to a population, wherein the population when fed a defined diet has a physiological response associated with variants (alleles) of at least one diet-regulated disease-associated gene listed in table 2 compared to the genotype (alleles).

A method for formulating a food beneficial to a population, wherein the population when fed a defined diet has a physiological response associated with variants (alleles) of at least one diet-regulated disease-associated gene listed in table 3 compared to the genotype (alleles) that produces a different response.

The invention includes other embodiments and applications of the data presented here which will become clear in view of the following detailed description and claims.

### DETAILED DESCRIPTION OF THE INVENTION

The previously-disclosed method for identifying diet-regulated disease-associated genes is set out below in PART 1. The new data is set out in PART 2.

PART 1: Previously-Disclosed Methods for Identifying Diet-Regulated

Disease-Associated Genes

A method for identifying diet-regulated disease-associated genes is as follows: Two different inbred genotypes (known genotypes) are selected (A and B). One of these genotypes (A) is more susceptible to a disease (can be any “undesirable” phenotype), and the other genotype (B) is less susceptible to the same disease. Then each genotype is divided into two groups (A1 and A2 and B1 and B2). For one genotype, each group is fed a different diet (A1 is fed diet No.1 and A2 is fed diet No.2, and similarly for B1 and B2). Gene expression is then compared across the strains that differ in either genotype or in diet, but not in both. I.e., A1 is compared with A2, and A1 is compared with B1; but A1 is not compared with B2. This allows the investigator to deal with only one variable at a time. Differential gene expression is identified between the compared groups, and genes are identified that show significant changes in expression (e.g., a 1.5 or 2.0 or 2.5-fold increase or decrease in gene expression). The genes so identified are diet-regulated disease-associated genes. As a further step, these identified genes are then compared against independently-identified diet-regulated and/or disease associated QTL's. This step helps add assurance to the identification and to help differentiate cause from effect for genes that are differentially expressed in response to diet.

More specifically, the method of the invention may be carried out as follows: a) comparing gene expression between two inbred strains in response to different diets, wherein one inbred strain is susceptible to a disease and the other inbred strain is not susceptible to the disease, b) identifying those differentially expressed polynucleotides that overlap with independently-derived diet-regulated QTLs, and c) analyzing the data to identify diet-regulated disease-associated polynucleotides. Generally the disease in question is a diet-associated disease. Gene expression is usually compared by comparing mRNA abundance (for example using a cDNA array), but may be compared by looking at protein levels. Often in-bred strains of mice are used mRNA abundance is compared between strains in response to different diets. Lastly, genes (or EST's or any polynucleotides) that have been identified as being significantly differentially expressed are compared with previously-identified, independently-derived diet-regulated QTLs. Various methods are also disclosed for use in disease screening, monitoring and treatment. The disclosed methods may also be used for formulating medical foods used to treat and prevent disease and slow disease progression. Arrays are also disclosed that employ one or more genes/polynucleotides identified by the method of the invention. Various new diet-regulated disease-associated genes and ESTs are also disclosed, as are compositions of medical foods and dietary supplements that have use for prophylactically treating populations and individuals susceptible to disease, or therapeutically treating populations and individuals who have disease.

Using the method of the invention, gene expression comparisons are made within a strain based upon differences in diet, and between strains fed the same diets. We can therefore identify genes regulated by diet in one or both strains, genes regulated by the one or both genotypes, and genes regulated by the interaction between diet and genotype. That is, certain genes will be regulated without regard to diet—the regulation of these genes will depend upon genetic makeup only. Other genes will be regulated in the same manner in all individuals in the population by what is eaten, although the level of the response may differ among individuals. The regulation of other genes will depend upon an individual's genetic makeup and how it responds to dietary variables. The regulation of these genes therefore differs between individuals even if they eat the same concentrations of dietary chemicals.

Since gene expression differences result from differences in DNA sequence (0.1% difference among humans), the methods of the invention can be practiced by associating genetic differences in the identified genes with disease incidence, severity, or progression. That is, single nucleotide polymorphisms (SNP) or other polymorphisms in the identified gene sequences can be used to identify the variants of diet-regulated genes that are associated with disease or that predict the severity of the disease once diagnosed. The method identifies genes whose abundance (or regulation) is affected by diet. However, a logical and obvious extension is that differences in protein or enzyme activities of these diet-regulated genes are also likely to influence disease development or severity. Analyzing SNPs or other polymorphisms in the promoter and gene may therefore be used in place of expression profiling.

Various embodiments of the invention include the following:

A method for determining the susceptibility of an individual to a disease, wherein said disease involves a diet-regulated disease-associated polynucleotide, the method comprising: screening an individual for the presence and/or expression of a plurality of polynucleotides identified by the method above or by associated polymorphisms in gene sequence, wherein the pattern of gene polymorphisms of and in said plurality of polynucleotides corresponds with the susceptibility of an individual to a certain disease.

A method for monitoring the progression of a disease in a subject, the method comprising: at a first date, screening an individual for the presence of gene variants of a plurality of polynucleotides identified by the method above that is associated with the incidence, severity, progression, or prognosis of a disease.

A method for treating a subject so as to reduce the risk of the individual developing a diet-associated disease, the method comprising: screening an individual for the presence and/or expression of a plurality of polynucleotides identified by the method above, wherein the pattern of gene variants of said plurality of polynucleotides corresponds with the susceptibility of an individual to a certain disease and reducing risk by altering diet in a defined manner.

A method for treating a subject so as to reduce the risk of the individual developing a diet-associated disease, the method comprising: screening an individual for variants of genes listed in Tables 2 and 3 and other publicly available genes associated with disease risk and altering the diet of the individual so as to reduce the risk of the subject developing the disease.

A method for treating a subject so as to ameliorate a diet-associated disease, the method comprising: screening an individual for variants (alleles) of a plurality of polynucleotides identified by the method above, wherein the pattern of variants (alleles) of said plurality of polynucleotides corresponds with the susceptibility of an individual to a certain disease; and altering the diet to change the progression of the disease.

A method for treating a subject so as to reduce the progression of a diet-associated disease, the method comprising: screening an individual for variants (alleles) of a plurality of polynucleotides identified by the method above, wherein the pattern of variants (alleles) of said plurality of polynucleotides corresponds with the susceptibility of an individual to a certain disease, and altering the diet of the individual so as to affect an improvement in the progression of the disease.

A method for identifying the suitability of various drugs or medical food regimens for a subject diagnosed with a disease, the method comprising: screening an individual for the presence of variants (alleles) regulated by diet, using the above methods.

A method for identifying genetic susceptibility of a subject to a chronic disease so as to select appropriate drug(s) or diets for reducing the incidence, severity, or progression of the disease or symptoms of the disease, the method comprising: screening an individual for the presence polymorphisms in genes regulated by diet, genotype, or their interactions using the above methods.

In all cases, the method claims the use of individual or combinations of diet-,genotype-, or diet/genotype-regulated genes for diagnostics and/or development of drugs.

The strategy described for identifying genes participating in disease processes uses both gene expression analyses in inbred strains of mice (susceptible to disease and not susceptible to disease) and publicly available QTL information.

Identification of Diet-Regulated Disease-Associated ESTs and Genes

A multi-step procedure was developed to identify genes regulated by dietary chemicals that participate in disease development.

1. Analyze mRNA abundance in inbred strains of mice in response to different diets using cDNA arrays (variations include measuring protein abundance). One strain of mice used is susceptible to a disease and the other strain is not be susceptible. Diets are chosen to induce chronic diseases such as obesity or diabetes.

2. Compare expression profiles between inbred strains of mice that differ in susceptibility to diet-induced disease. Differences in mRNA abundance identify genes regulated by genotype, diet, and their interactions. Since the diet is chosen to induce disease development in susceptible genotypes, a subset of these genes will be involved in disease development.

3. Differentiate between cause from effect genes by determining the map position of diet-regulated to independently-derived QTLs. Differentially expressed genes that overlap QTLs become candidate genes for the disease (an example of association mapping [16]).

4. Characterize the expression and activity of a subset of the genes and proteins that were identified by expression array technology and QTL analyses in animal models of the disease

5. Identify polymorphisms in candidate disease genes

6. Examine their associations in humans who are healthy vs. those showing symptoms of the disease.

Detailed Methodology

Animals, Diets and Protocols.

Male or virgin female (eliminates complications and effects of pregnancy) mice of defined genotype are fed a semi-purified diet containing 4% corn oil for 1 wk and then randomly assigned to control or experimental diets for at least 2 wks and up to the normal lifespan of the mouse. Each diet contains 1.4% of the respective total oil content as soybean oil to assure adequate fatty acid content (NRC 1995, [27]). An example of the diets is shown in Table 2. The diets are formulated according to modified AIN-76 guidelines (NRC 1995,[27]), were pelleted, and color coded by a commercial vendor. The diets are balanced for minerals, vitamins, fiber, and protein, and differ in carbohydrate and lipid level. In some cases, individual chemicals (natural or man-made) can be added to the diet to determine their effect on gene expression.

Mice are caged and fed individually with free access to food and distilled water in temperature-controlled rooms maintained at 23±1° C. with a 12-h light:dark cycle. Animal care meets National Institutes of Health guidelines. Food spillage is also monitored throughout the course of the experiment. Efficiencies of energy utilization are calculated from the recorded weekly weight gain/calculated weekly energy intake.

At the end of the feeding period, all mice were deprived of food for 12 h and offered a preweighed 3-g pellet of their assigned diet. After 2 h, the uneaten food was removed; at a defined time after, all mice were injected intramuscularly with 0.02 mL/g body weight of ketaset/xylazine mixture (Ketaset, Fort Dodge Laboratories, Ft. Dodge, Iowa) for collection of blood via cardiac puncture. Immediately thereafter, they are killed by cervical dislocation and their livers and hearts removed, individually frozen in liquid nitrogen, and stored at 80° C. for mRNA isolation. Altering the length of time for depriving food, for length of feeding, and for time to collection would all be trivial and obvious modifications of this protocol.

PART 2: New Data

New data has been produced by use of the methods previously described in the parent patent application, and by new analysis of data previously obtained. This new data reveals previously-known genes that have been newly-identified as being diet-regulated and disease-associated.

Analysis of Images. Phosphorimager data were submitted to Genome Systems for scanning and analysis using their internal software. All numerical data generated were returned to us. Background correction was calculated for each spot by subtracting the pixel density value of neighboring pixels from the pixel density within the spot. Spot intensity values were the background corrected mean pixel density for each spot multiplied by the number of pixels comprising the spot. For each pair of filters (A and B), spot intensity values were normalized using a ratio of background corrected global mean intensities. Global means were calculated for all spot intensity values on each filter (mean_{A} and mean_{B}). All spot intensity values on filter B were multiplied by a ratio of mean_{A}/mean_{B}. For each clone, spots were arrayed in duplicate on each filter. The intensity value for calculating the expression ratio was the values of those normalized for each pair. Genome Systems performed these calculations.

Data from replicates were averaged and then analyzed using the method described in study of gene expression in obese and diabetic mice [37] as modified by Lin et al [45]. This procedure detects genes whose expression does not change from genes whose expressions change by assessing their differential expression relative to the intrinsic noise found in the nonchanging genes. We report the results of these statistical analyses in this paper.

The Method of Analysis was as Described Above

Analysis of Images. Phosphorimager data were submitted to Genome Systems for scanning and analysis using their internal software. Background correction was calculated for each spot by subtracting the pixel density value of neighboring pixels from the pixel density within the spot. Spot intensity values were the background corrected mean pixel density for each spot multiplied by the number of pixels comprising the spot. For each pair of filters (A and B), spot intensity values were normalized using a ratio of background corrected global mean intensities. Global means were calculated for all spot intensity values on each filter (mean_{A} and mean_{B}). All spot intensity values on filter B were multiplied by a ratio of mean_{A}/mean_{B}. For each clone, spots were arrayed in duplicate on each filter. The intensity value for calculating the expression ratio was the values of those normalized for each pair. Genome Systems performed these calculations.

Data from replicates were averaged and then analyzed using the method described in study of gene expression in obese and diabetic mice [53] as modified by Lin et al [63]. This procedure detects genes whose expression does not change from genes whose expressions change by assessing their differential expression relative to the intrinsic noise found in the nonchanging genes. Results are given below.

Bioinformatics. Accession numbers of each clone were converted to NCBI's unique identifier number, GI. FASTA sequences of the 354 genes were blasted against the GenBank database. The accession or GI numbers and the function of each gene were then used to search the Mouse Genome Database at Jackson Laboratory for chromosomal map positions. Links within MGD were used to find additional functional information in Locus Link (NCBI) and the Online Mendelian Inheritance in Man (OMIM) databases, since some genes have different names in different databases. Primary literature reports were found for genes mapping to diabesity QTL. Genes mapping to obesity QTL are provided in Table 3. Some information regarding gene function therefore comes from diverse cells, tissues, and organisms.

Results

Expression levels of 18,000+ genes were compared among livers of each genotype fed AL or CR and between genotypes (A/a cf A^{vy}/A) fed either AL or CR. Genotypes were confirmed by amplification of an A^{vy} specific fragment of 870 bp (FIG. 1). These comparisons are designated Aal:Yal [A/a fed AL divided by A^{vy}/A fed ad libitum (AL) with caloric restriction (CR)] and are reported in Expression Ratio columns in Tables 1-3. We analyzed expression of only those genes whose expression levels showed statistical significance based upon the algorithm of Lin et al [53, 63]. As calculated, the ratios do not show up- or down-regulated genes but rather refer to the comparison between the two genotype:diet cf genotype:diet combinations. For these criteria, the four two way comparisons between A/a and A^{vy}/A fed 70% or 100% of ad libitum caloric intakes revealed 388 genes whose regulation differed among various genotypes and diet combinations. Of these 388 genes, the functions of 223 are of known function, are named genes (see Table 1), or whose map position in mouse chromosomes are known. The genes were sorted based upon gene ontology into metabolic enzymes, signal transduction, structural, transcription, splice components, immune function, protease and unknown function (FIG. 2 and Table 1).

Genes Regulated by Genotype

The sum total of gene expression of all genotype-regulated genes contributes to physiological differences between A^{vy}/A and A/a mice. Ten (10) genes were regulated by genotype regardless of caloric intake: A/a fed CR relative to A^{vy}/A fed CR and A/a fed AL relative to A^{vy}/A fed AL (Table 1, genotype in both diet rows). Ectopic expression of agouti altered abundance of these genes in a predictable manner: 7 genes more abundant in A^{vy}/A and A/a mice and the other 3 were less abundant in both genotypes. An additional 76 genes were regulated by genotype (comparing abundance of mRNA in A/a vs A^{vy}/A in either AL fed mice (Table 1, Genotype AL, 39 genes) or CR fed mice (Table 1, Genotype CR, 37 genes). These genes might be regulated in the same fashion regardless of calories eaten, but they did not pass the statistical cut off.

Several notable examples illustrate how ectopic expression of agouti (in A^{vy}/A) alters expression and phenotype:

Aes, amino-terminal enhancer of split is more abundant in A^{vy}/A relative to A/a mice regardless of the calories consumed. Aes is a co-repressor of NFκβ [96], which is activated in insulin resistant tissues. Aes is less abundant in muscle tissue of humans with a family history of T2DM relative to those with no family history (Table 2, [74]).

Mark4, MAP/microtubule affinity-regulating kinase is more abundant in A^{vy}/A relative to A/a mice regardless of the calories consumed. Mark4 participates in the Wnt/β-catenin signaling pathway [44], which, when misregulated, may result in cancer. GSK-3β (glycogen synthase kinase 3β) is a negative regulator of this pathway and is downregulated when cells are exposed to Wnts (rev. in [16]). GSK-3 is also a suppressor of glycogen synthase and insulin receptor substrate 1 [40].

Genes regulated by genotype in one dietary condition (Table 1, Columns 3 and 4) also contribute to the overall expression of A^{vy}/A phenotypes.

Diet-Regulated Genes

More genes were differentially regulated by diet in the A^{vy}/A genotype than in the A/a genotype. We previously showed that CR abolished metabolic efficiency in obese yellow (A^{vy}/A) mice [112]. Gene products with differential abundance in A^{vy}/A fed AL vs CR (Tables 1-3) may alter energy metabolism during caloric restriction. Among the genes regulated by diet are:

Pdtgs, Prostaglandin D Synthase, was more abundant in A^{vy}/A fed AL relative to A^{vy}/A fed CR. This enzyme produces prostaglandin G2 which is a precursor to prostaglandin J₂ (PGJ₂). PGJ₂, which is in turn a precursor to 15-deoxy-Δ¹²⁻¹⁴-PGJ2, the primary ligand of PPAR-γ (rev. in [69]).

Pparbp, Peroxisome proliferator activated receptor binding protein, is also more abundant in A^{vy}/A fed AL relative to A^{vy}/A fed CR. Pparbp is a coactivator of PPAR-α, -γ, retinoic acid receptor-α (RAR-α), retinoic X receptor (RXR), estrogen receptor (ER), and thyroid receptor β1 (TR-β-1) [118]. PPAR-γ appears to be one of the key regulators of glucose and lipid homeostasis [28].

Fabp, fatty acid binding protein, is more abundant in A/a mice fed ad libitum relative to A/a mice fed CR. Fabp may function by targeting its ligand to the nucleus and may participate in regulation of gene expression by binding to PPAR-γ [114].

Table 1 lists the other genes of known or predicted function, whose transcription is modulated by diet and genotype, that may also contribute to differences in energy metabolism between AL and CR A^{vy}/A. Integrating these genes into a coherent explanation for those differences may require analyses of gene expression with more mice of each group to improve statistical significance.

Genotype X Diet Interactions Genes

A subset of gene products (19 out of 223) identified by comparing A^{vy}/A vs A/a and calories eaten were regulated by more complex genotype X diet interactions: i.e., they were regulated by different genotypes fed CR or AL and by different diets in A/a vs A^{vy}/A mice (Table 1, rows 6). The majority of these genes (16) were regulated in the same manner in two different diet-genotype conditions: they were more abundant in A/a mice fed AL vs CR and in A^{vy}/A mice fed CR relative to A/a mice fed CR. Increased calories or the presence of the A^{vy} allele may independently contribute to increased transcription of these genes.

Genes Mapping to Diabesity QTL

The map positions of the 223 genes of known functions were determined and compared to QTL associated with various subphenotypes of diabetes (Table3). Twenty-eight (28) of the diet-, genotype-, and diet X genotype-regulated genes in liver mapped to diabesity QTL (Table 2). An interval distance of ±10 cM from a given QTL was used, a distance consistent with the marker density employed in most QTL association studies. Five murine QTLs involved in diabesity, insulin levels, or regulation of insulin like growth factor levels (labeled with footnotes 4 through 8 in Table 2) overlapped with T2DM QTLs in humans [21].

Several of the gene products regulated by diet, genotype, or their interactions are associated with diabetes and/or are in pathways that alter phenotypes consistent with one of the conditions of diabetes (Table 2, Diabesity Association). Genes mapping to diabesity QTL and differently regulated between AL fed A^{vy}/A and A/a mice (Aal:Yal column in Table 2) are likely to cause differences in diabetes subphenotypes observed in this model: e.g., continuous ectopic expression of agouti may “override” normal regulation of these genes:

Aes (43cM) maps near an insulin-like growth factor binding protein (Igftbp3q2) QTL (46 cM) on chromosome 10. IGF and its binding proteins, particularly IGFBP3, are thought to be involved in glucose homeostasis (rev. in [35]). Aes (amino-terminal enhancer of split) is a co-repressor of NF-κB, which is activated in insulin resistant tissues. Aes expression is increased in A^{vy}/A mice relative to A/a mice regardless of the diet (Table 2, columns 3 and 4). Aes expression in muscle was decreased significantly (p=0.0178) in individuals with a family history of T2DM relative to individuals with no family history [74]. However, its abundance was elevated (not statistically) in patients with DM. Evans et al [17] suggested that oxidative and stress-activated signaling pathways (e.g., NF-κB) underlie the development of complications in T2DM.

Pdgfra, platelet derived growth factor receptor α, was more abundant in A^{vy}/A mice fed AL relative to A/a mice fed AL (Table 2) and maps within a diabesity locus (Dbsty2) on chromosome 5. Dbsty2 is associated with increased adiposity. Pdgfra interacts with the Hedgehog signaling pathway. Changes in the hedgehog (Hh) pathway affected insulin production in the pancreas [99]. Expression of the receptor's ligand, Pdgfa, is under the control of Kruppel-like factor 5 (KLF5) and is cooperatively activated by the NF-κB p50 subunit [1]. A similar Kruppel-like gene, Klf1, is more abundant in A^{vy}/A mice relative to A/a mice regardless of the diet (Table 1).

Grb2, growth factor receptor bound protein 2, was less abundant in A^{vy}/A mice fed AL relative to A/a mice fed AL. Grb2 is a signal transduction adaptor protein that is recruited to caveolae-localized receptor complexes (including the insulin receptor) by increased levels of IRS-1 and insulin [5]. Grb2 participates with Ash to reorganize the cytoskeleton in response to insulin [101]. Grb2 maps to Chr. 11, 72cM near Nidd4 at 68 cM.

Several other genes had more complex regulatory patterns but may play a role in causing differences in subphenotypes of diabesity

Smo, smoothened homolog, is a member of the Indian Hedgehog (IHH) signaling pathway. Smo maps (Chr 6, 7.2 cM) near Fglu (Chr. 6, 16) which is associated with increased fasting plasma glucose levels. IHH may be involved in chronic pancreatitis and insulin production [45]. Smo mRNA was more abundant in A/a mice fed AL relative to A/a mice fed CR and was more abundant in A^{vy}/A mice fed AL relative to A/a mice fed reduced calories, an example of diet X genotype interaction.

Flnc, filamin also mapped (8.5 cM) near the Fglu QTL on chromosome 6. Filamin had the same complex expression pattern as Smo. Insulin causes changes in cytoskeleton architecture [101] and filamin may bind to the insulin receptor [31].

Some individual genes in Table 2 have not been studied for a role in processes affected by diabetes, but members of their functional family have been linked to processes that are altered in that disease. Other genes and their products mapping to diabesity QTL have only a tentative association to conditions in diabetes—Madl2 (mitotic arrest deficient), debrin, and disheveled 2. Nevertheless, these genes are candidates for diabetes subphenotypes in A^{vy}/A mice by virtue of their regulation by genotype or diet and their map position near QTL associated with diabetes symptoms.

Genes Mapping to Obesity QTL

Forty-one (41) genes mapped to weight gain or obesity QTLs (Table 3) and 12 of these genes (Idh1, Pdgfra, Flnc, Dvl2, Nhp2, Sept8, Skpa1, Mpdu1, Grb2, Gsc, Gpt1, H1fo) mapped to overlapping diabesity QTLs. Genes mapping to obesity loci may contribute to the subphenotypes expressed in this model (Table 3). Other QTL for adiposity at various sites and total carcass lipid levels (for a comprehensive review, see [6]) were not included in our analyses since these parameters were not measured in this study. Many diabesity QTL overlap obesity QTL (Tables 2 and 3) as would be expected for the diabesity phenotype [60]. Associations with many specific molecular pathways influencing or involved in obesity and/or weight gain can be made for each of the other diet-, genotype-, and diet X genotype-regulated genes mapping to obesity and weight gain QTLs (Table 3, Obesity Association).

Discussion

Diabesity is a complex trait resulting from interactions between multiple genes and environmental factors. In humans, chronic exposure to excessive calories, deficiencies of micronutrients, and certain types of macronutrients induce obesity and diabetes in individuals, presumably without deleterious mutations in participating genes. These diseases therefore fit the common variant/common disease hypothesis proposed by Lander [54] and Collins and colleagues [11]. We have proposed that one or more of the gene products participating in development of chronic diseases will be regulated at least in part by diet ([43, 72] and rev. in [41]) since different macronutrients and excess calories are associated with almost all chronic diseases (e.g., [109, 110]).

Although chronic diseases are multigenic in nature, much information regarding the pathways involved in disease development has been discovered by the study of rodent models with single gene defects or induced mutations (knockouts and transgenics) that mimic diabetes and/or obesity. Comprehensive reviews of the mouse models for insulin resistance [36] and obesity have recently been published [6]. The general conclusion from these reviews echoes the conclusion of Wolff [111] that similar if not identical phenotypic expressions of a disease state can be reached by different metabolic routes. That is, alterations in many pathways can produce the same phenotype. The specific genes and their transcriptional regulation reported herein, therefore, are most applicable to obesity and subphenotypes of diabetes produced by the dominant mutation (A^{vy}) in the agouti gene. Nevertheless, these genes and their variants may identify sets of pathways that collectively produce the specific diabetes subphenotypes and obesity pattern in A^{vy}/A mice or other genetically-susceptible mice. That is, some of these pathways may also be involved in other models of diabesity if gene variants in the key regulatory or structural genes collectively produce expression changes similar to those observed in this specific mouse model. Genes identified in the A^{vy}/A and A/a comparison may contribute to obesity or diabesity in humans if their regulation is altered in a similar manner. Five of the genes found in our analyses (fatty acid synthase, malate dehydrogenase, sterol C5 desaturase, dynein, and epidermal growth factor receptor pathway 15) were also differently regulated in livers of BTBR-ob/ob (obese and diabetes susceptible) compared to C57BL/6-ob/ob mice (obese and diabetes resistant) fed a chow based diet ad libitum. Although we and others identify candidate disease genes through gene expression analyses, changes in the activity of proteins encoded by coding SNPs may also be associated with disease development [54].

Laboratory animal studies have consistently shown that reducing caloric intake is the most effective means to reduce the incidence and severity of chronic diseases, retard the effects of aging, and increase genetic fidelity (rev in [103, 107]). Caloric restriction may produce its largest effects by increasing respiration [62] with the concomitant increase in the amount of NAD⁺ [61]. NAD also is a cofactor for Sir2, a histone deacetylase involved in chromatin silencing of nucleolar rDNA and the telomere-located mating type locus [27, 67]. Several genes involved in NAD+ metabolism were found in our screen, (e.g., NADH-ubiquinoone oxidoreducatase 1α subcomplex, lactate dehydrogenase, malate dehydrogenase, aldehyde dehydrogenase, and isocitrate dehydrogenase) but these were not regulated consistently within any one genotype x diet condition (Table 1).

Several laboratories examined the effect of caloric restriction on gene expression in individual mouse strains in relation to their age. Genes involved in many different pathways were regulated by CR and/or aging in livers of C3B19RF₁ (a long-lived F₁ hybrid mouse [15]) and C3B10RF₁ mice [7], in muscle of C57BL/6 mice [57, 58, 106], in heart tissue from B6C3F₁ mice [56], and in livers of the long-lived Snell dwarf (dw/dw) stock. A few genes identified in these studies (Aes, Fasn, Fabp, [7]) matched genes found in Table 1 although the regulation by CR was not always consistent with our results.

We found 388 hepatic genes or ESTs regulated in the same manner in replicate experiments with 223 genes having a known function. We believe that the designations of genotype, diet, and genotype X diet interactions will be specific to the experimental model used in this study. Agouti protein has been shown to regulate gene expression in cell culture systems [24, 25, 93]. Therefore, in the A^{vy} genotype, the constant ectopic expression of agouti signaling protein may “override” normal genotype-specific or diet-regulated gene regulation. Less obvious will be those genes regulated by genotype X diet (i.e., environmental interactions). Many promoters are regulated by multiple receptors and by accessory factors. For example, HNF3γ is regulated differently in rats fed protein-free, casein, or gluten diets [37]. Hence, depending upon the diet, transcription of genes regulated by this receptor may show differential regulation by non-diet influenced factors in mice fed diets that decrease the expression of Hnf-γ. A confounding variable that is likely to alter gene regulation will be variants (SNPs) within each regulatory gene and the promoters that interact with them [116]. Although this complexity is noteworthy, recent reports from the human genome project suggest a limited number of haplotypes in the human population [26] and it is likely that mice also have a limited genetic diversity [104].

The sum total of the expression differences between A^{vy}/A and A/a mice fed AL identify the hepatic genes that contribute to the obese yellow phenotype (Aal:Yal column under Expression Ratio Table 1). Genes of all functional classes and types of regulation were differently expressed in this genotype comparison. No apparent pattern was discernable within this set and new analytical tools will be needed to identify key regulatory and expression patterns among the many genes, their pathways, and their type of regulation.

QTL are used to associate chromosomal regions with complex traits. There are now over 1700 QTL for disease, subphenotypes of disease, enzyme or protein levels, behavior, and other complex traits in mice. The limitations of using QTL data are that (i) they may be specific to the inbred strains analyzed, (ii) identify 20-30 cM regions of DNA, and (iii) often can not detect interactions with other loci [20]. In addition, few mapping studies rigorously control or report diets; environment is known to have a large influence on the identification of QTL affecting complex traits, at least in plants [73]. Our approach combines the strength of array technology with the power of genetics to identify potential causative genes. A key additional component of our approach is the rigorous control of diet composition and a timed feeding regimen [71, 72] that will allow for replication of the experiments.

Even with limitations of the current experiment (type of array, number of mice, single tissue source), the data presented herein identify potential novel candidate genes in many different functional pathways that may play a role in expression of subphenotypes of diabesity. Several of the genes that were found to be diet-regulated and mapped to diabetes QTL had previously been linked to specific pathways affected by or involved in diabetes. Aes, Grb2, and Pdgt1 are linked to Type 2 diabetes or are in pathways directly regulating insulin function. Other genes mapping to QTL from our screen can be associated with various alterations in metabolism found in diabesity. They become candidates for further testing.

Since obesity is an “amorphous” phenotype, with adiposity, weight gain, and overall weight as the key phenotypic markers, it is more difficult to compare candidates identified in this screen with those found in other model organisms or humans (rev in [94]). In addition, Wolff reviewed phenotypic and molecular differences between obesity induced by dominant mutations in the agouti gene and by the recessive mutation Le^{ob} in the leptin gene and concluded that many physiological parameters are diametrically opposed in these two obesity models [111], a conclusion consistent with that of others [6, 76]. Genetic analyses support this conclusion since a large number of overlapping obesity and weight gain QTL have been identified (see Table 3 for a subset of these QTL. Nevertheless, genes analyzed in this screen that are regulated by diet, genotype, and genotype X diet that map to obesity QTL may be considered candidates for obesity development or severity.

### SUMMARY

The strategy described is a means to identify diet-, genotype-, and genotype X diet-regulated genes that cause or promote the development and severity of complex phenotypes. A similar approach compared gene expression patterns in strains of nondiabetic obese mice and diabetic mice but did not systematically alter diet [53]. Comparative genetic approaches can be applied to different mutant models and their normal inbred parent or strain and to congenic siblings produced specifically for separating and combining QTL producing a complex phenotype (e.g., [81]). By comparing across the different genotypes fed the same diet, genotype-regulated genes can be identified. Similarly, by feeding two or more diets to mice with different genotypes, diet-regulated- and diet X genotype-regulated genes can be identified.

Understanding diabetes and obesity will require integration of knowledge from individual pathways that have been elucidated to date. However, inclusion of diet as a variable in a systems biology approach will also be necessary to fully explain complex phenotypes, almost all of which are influenced by environment, and specifically by dietary variables. This type of scientific study is called nutrigenomics or nutritional genomics (rev. in [42]). Knowledge of the interactions of diet and genotype will be needed when testing and treating these diseases in human populations.

TABLE 1
Genes Regulated by Diet-, Genotype-, and Diet X Genotype¹

| | | | | Aal_vs_Acr | | Yal_vs_Ycr | | Aal_vs_Yal | | Acr_vs_Ycr | |
|---|---|---|---|---|---|---|---|---|---|---|---|
| GI # | MGI # | Locus | Name/Function | Ratio | P value | Ratio | P value | Ratio | P value | Ratio | P value |

Diet-Regulated in Genotype (A/a)

| | | | | | |
|---|---|---|---|---|---|
| 1531373 | MGI:290657 | 2810404F18 | PAK-box/P21 Rho binding | 14.50 | 0.0068 |
| 1918428 | MGI:469298 | Aldo3 | Aldolase 3, C isoform | 3.63 | 0.0002 |
| 1796404 | MGI:390623 | Bsp | Brain specific protein | 4.74 | 0.0562 |
| 2187093 | MGI:511045 | Bst1 | Bone marrow stromal antigen | 0.04 | 0.0001 |
| | | | NAD+ nucleosidase activity | | |
| 1902608 | MGI:443722 | C3ip1 | Kelch-like protein; actin organizer | 3.20 | 0.0151 |
| 1436232 | MGI:260125 | Cacna2d2 | Ca²⁺ channel protein α-2 | 3.19 | 0.0135 |
| 14210850 | MGI:88356 | Cdh3 | Cadhedrin3 | 2.97 | 0.0703 |
| 1917726 | MGI:463219 | Ceacam1 | CEA-relateded cell adhesion molecule | 5.54 | <0.0001 |
| 1542193 | MGI:255279 | Cenpb | Centromere autoantigen B | 3.60 | 0.0014 |
| 18623506 | | Dntnp | Dosal neuron-tube nuclear protein | 7.96 | 0.0066 |
| 1882541 | MGI:426105 | Dolpp1 | Dolichyl pyrophosphate (Dol-P-P) | 3.85 | <0.0001 |
| | | | phosphatase | | |
| 1861663 | MGI:421248 | Es22 | Liver carboxylesterease | 15.61 | 0.0173 |
| 1875855 | MGI:419365 | Fabp2 | Fatty acid binding protein, intestinal | 13.85 | 0.0497 |
| 2259449 | MGI:523407 | Idh1 | Isocitrate dehydrogenase | 4.68 | <0.0001 |
| 1915278 | MGI:109368 | Imap38 | Immunity associated protein 1 | 3.16 | 0.0457 |
| 2306066 | MGI:1100848 | Kpna4 | Karyopherin (importin, nucleus) | 7.76 | 0.0022 |
| 1326737 | MGI:1353635 | Lmcd1 | LIM (Zn finger) and cysteine-rich | 3.48 | 0.0006 |
| | | | domains 1; Dyxin | | |
| 1650973 | MGI:337853 | Madd | MAP kinase activating death domain | 4.77 | <0.0001 |
| 1514097 | MGI:284915 | Map3k7 | Tak1, TGF-beta activated kinase | 3.04 | 0.0419 |
| 1711859 | MGI:358044 | Mdm2 | Double minute 2 | 3.36 | 0.0009 |
| 2050049 | MGI:426906 | Pfkb1 | Fructose 2,6-bisphosphate 2- | 3.61 | 0.0002 |
| | | | phosphatase | | |
| 54005 | MGI:98105 | Rps12² | 40S ribosomal protein S12 | 11.36 | 0.08 |
| 1494419 | MGI:277834 | Rtp801 | RTP801; REDD1 - Hypoxia inducible | 0.05 | 0.0025 |
| | | | factor responsive protein | | |
| 1806780 | MGI:98283 | Sfrs1 | Splicing factor, arg/ser-rich 1 | 0.29 | 0.0058 |
| | | | (ASF/SF2) | | |
| 1487360 | MGI:273396 | Slc7a8 | Solute carrier cationic amino acid | 3.10 | 0.0152 |
| | | | transporter | | |
| 1356860 | MGI:229093 | Smurf1 | Smad ubiquination regulatory factor | 0.22 | 0.0479 |
| 1530059 | MGI:98387 | Spnb1 | β-spectrin | 2.88 | 0.0286 |
| 30705079 | MGI:1858416 | Stk39 | Ser/Thr kinase 39, STE20/SPS1 | 4.76 | 0.0026 |
| 1699544 | MGI:361386 | Tbl1x, Tbl2 | β transducin | 5.04 | 0.002 |
| 1505034 | MGI:278870 | Tln | Talin | 3.38 | 0.076 |
| 1725397 | MGI:357732 | Tnfrsf7 | CD27L receptor; TNF □ receptor | 2.89 | 0.0361 |
| 1294292 | MGI:217897 | Zip4 | ZN transporter | 3.12 | 0.0052 |

Regulated by Diet in Genotype (A^{vy}/A)

| | | | | | |
|---|---|---|---|---|---|
| 1862752 | MGI:431225 | 3110004L20Rik | Sugar and other transporter | 2.05 | 0 |
| | | | domains | | |
| 2235102 | MGI:532359 | Ak1 | Adenylate kinase 1 | 5.62 | 0.0005 |
| 1504171 | MGI:99600 | Aldh2 | Acetaldehyde Dehydrogenase II | 0.52 | <0.0001 |
| 2256323 | MGI:522039 | Ampd2 | AMP deaminase 2 | 0.63 | 0.0066 |
| 1643104 | MGI:88051 | Apoa4 | Apolipoprotein A-IV | 0.64 | 0.0558 |
| 1862743 | MGI:431204 | Arpc1b² | Actin related protein 2/3 | 1.64 | 0.0016 |
| | | | subunit 1b | | |
| 1655040 | MGI:347017 | Apo5b² | ATP synthase H+ beta subunit | 1.70 | 0.0006 |
| 1863019 | MGI:1096327 | Axin | Signaling, interacts w/GSK-3 | 1.62 | 0.0084 |
| 1756557 | MGI:378740 | B230106l24 | Lipolytic enzyme (esterase, | 16.57 | 0.0128 |
| | | | lipase, thioesterase) | | |
| 1428458 | MGI:258273 | Bad | Bcl-associated death promoter | 3.62 | <0.0001 |
| 31542013 | MGI:1924832 | Bb1 | Membrane bound acyl | 1.62 | 0.0037 |
| | | | transferase | | |
| 2157574 | MGI:2446213 | BC013712 | Icb-1 (basement membrane | 0.65 | 0.039 |
| | | | induced gene) | | |
| 1904205 | MGI:2652892 | BC038156 | BC038156 | 1.57 | 0.0452 |
| 29612642 | MGI:2443590 | BC049929 | Helicase c2 | 5.93 | 0.0038 |
| 1738624 | MGI:1914368 | Bfar | Bifunctional apoptosis regulator | 0.58 | 0.0004 |
| 1316779 | MGI:88251 | Calm1 | Calmodulin 1 | 0.63 | 0.0106 |
| 1752088 | MGI:376580 | Cd2bp2 | CD2 antigen binding protein | 1.67 | 0.0005 |
| 2199793 | MGI:1918341 | Cdkl1 | CDC2 related kinase (several in | 0.64 | 0.0515 |
| | | | mouse genome) | | |
| 2283495 | MGI:546651 | Cib1 | Calmyrin - Ca²⁺ and integrin | 2.14 | <0.0001 |
| | | | binding 1 | | |
| 1876797 | MGI:418939 | Cth | Cystathionine γ - lyase | 0.65 | 0.053 |
| 2196076 | MGI:501072 | Cttn | Cortactin - oncogene | 1.68 | 0.0003 |
| 1661553 | MGI:345223 | Cyp4a10² | Cytochrome P450 IVA1 | 0.62 | 0.0084 |
| 1514357 | MGI:1859320 | Cyrh1 | Cysteine and histidine rich 1 | 2.59 | <0.0001 |
| 2247456 | MGI:505835 | D6Wsu176e | Predicted osteoblast protein | 0.63 | 0.0271 |
| 2164189 | MGI:509277 | Eps15^{23,3} | Epidermal growth factor | 1.63 | 0.0055 |
| | | | receptor path - 15 | | |
| 1902698 | MGI:442687 | Ets1 | Transcription | 2.45 | <0.0001 |
| 1671526 | MGI:351529 | Fasn³ | Fatty acid synthase | 1.56 | 0.0606 |
| 1901893 | MGI:102779 | Fen1 | Flap endonuclease | 0.61 | 0.0036 |
| 2250182 | MGI:534640 | Fgf1 | Fibroblast growth factor 1 | 1.59 | 0.0102 |
| 1661575 | MGI:345702 | Fhod1 | Rac1 GTPase effector FHOS | 0.53 | <0.0001 |
| 2157916 | MGI:500651 | Gstm1 | Glutathione S transferae mu | 1.60 | 0.006 |
| 2258877 | MGI:523960 | H1f0 | Histone H1 | 0.33 | 0.0001 |
| 54930 | MGI:95950 | H2-T18 | T-haplotype-specific | 0.58 | 0.0001 |
| | | | elements (ETn related) | | |
| 1756739 | MGI:96157 | Hmgb2 | HMG-Box containing protein 2 | 2.14 | <0.0001 |
| 1915022 | MGI:455872 | Hspb7 | Heat shock protein, member 7 | 0.58 | 0.0001 |
| 1841225 | MGI:1917065 | Kcp3 | Keratinocytes associated | 0.21 | <0.0001 |
| | | | protein 3 | | |
| 2187569 | MGI:96759 | Ldh1 | Lactate dehydrogenase | 1.71 | 0.0004 |
| 2248217 | MGI:507516 | Lims2 | Pinch protein - Adhesion | 0.48 | <0.0001 |
| | | | function | | |
| 1861860 | MGI:421291 | Magel2 | Mage-g1 = melanoma antigen, | 0.55 | <0.0001 |
| | | | family 2 | | |
| 2248065 | MGI:507446 | Man1b | Mannosidase 1 β | 0.39 | <0.0001 |
| 1909953 | MGI:453942 | Maz | MYC-associated Zn finger | 0.50 | <0.0001 |
| 1902178 | MGI:442591 | Mdh2^{2,3} | Malate dehydrogenase | 1.59 | 0.0106 |
| 2081170 | MGI:2671945 | Mdp77 | Muscle-derived protein variant 2 | 3.48 | 0.0146 |
| 1539394 | MGI:246987 | MGC7259 | Zn-finger RING - mRNA | 2.93 | <0.0001 |
| | | | turnover and process | | |
| 2196374 | MGI:1928139 | Mrps10 | Ribosomal protein S10 | 1.58 | 0.0135 |
| 1876496 | MGI:418997 | Msl31 | Male specific lethal homolog | 0.58 | 0.0001 |
| 2192570 | MGI:512345 | Mtmr1 | Myotubularin - tyrosine protein | 0.60 | 0.0006 |
| | | | phosphatase | | |
| 1749045 | MGI:378989 | Nck | Cytoplasmic protein | 1.68 | 0.0004 |
| 2158221 | MGI:500438 | Neu1 | Neuraminidase 1 | 1.95 | <0.0001 |
| 2292294 | MGI:545881 | Nhp2 | Nucleolar protein family A,, 2 | 1.98 | <0.0001 |
| 1863483 | MGI:431735 | Nid1 | Entactin, glycoprotein | 2.86 | <0.0001 |
| | | | membrane | | |
| 1464021 | MGI:263469 | Nmyc1 | Oncogene | 1.77 | 0.0223 |
| 2157584 | MGI:508869 | Npr3 | Natriuretic peptide receptor 3 | 0.62 | 0.0117 |
| 2057408 | MGI:1352466 | Nr2c2 | CD-1 orphan receptor TAK | 0.47 | <0.0001 |
| 2200234 | MGI:514479 | Ocdcp | Ornithine decarboxylase like | 3.20 | <0.0001 |
| 1317041 | MGI:222839 | Ocilrp1 | Osteolast inhibitory lectin | 0.55 | <0.0001 |
| | | | related | | |
| 1486101 | MGI:274247 | P4ha1 | Procollagen-proline 2- | 3.31 | 0.0001 |
| | | | oxoglutarate 4-dioxygenase | | |
| 1755663 | MGI:378261 | Palmd | Palmdelphin | 0.44 | <0.0001 |
| 1875271 | MGI:1330223 | Papss2 | 3′-phosphoadenosine 5′- | 0.48 | <0.0001 |
| | | | phosphosulfate synthethase 2. | | |
| 1862676 | MGI:431447 | Parn | Poly(A)-specific ribonuclease c | 1.83 | <0.0001 |
| 1863752 | MGI:430049 | Parva | Parvin | 1.90 | <0.0001 |
| 1767550 | MGI:376083 | Pea15 | Phosphoprotein enriched | 1.52 | 0.0909 |
| | | | in diabetes | | |
| 1902592 | MGI:443675 | Pfdn4 | Prefoldin | 1.85 | <0.0001 |
| 2259380 | MGI:523448 | Ppm1b | Protein phosphatase 1B, Mg²⁺, β | 0.57 | 0.0001 |
| 1660308 | MGI:346269 | Ptgds | Prostaglandin D synthase | 4.00 | <0.0001 |
| 2200347 | MGI:514164 | Ptprg | Protein tyrosine phosphatase γ | 1.73 | 0.0002 |
| 2247233 | MGI:505787 | Rnpep | Arginyl aminopeptidase | 0.64 | 0.0483 |
| 1876782 | MGI:418889 | Sc5d³ | Sterol-C5-desaturase | 0.61 | 0.0013 |
| 1863123 | MGI:431472 | Scamp2 | Secretory carrier membrane | 1.82 | <0.0001 |
| | | | protein 2 | | |
| 25990187 | MGI:1100846 | Pparbp | Peroxisome proliferator | 2.72 | 0.0001 |
| | | | activated receptor binding | | |
| | | | protein | | |
| 1915208 | MGI:457259 | Siat9 | Sialytransferase 9 (CMP-NeuAc- | 0.27 | 0.0116 |
| | | | lactosylceramide) | | |
| 2201329 | MGI:479801 | Skpa1 | S-phase kinase associated | 0.60 | 0.0015 |
| | | | protein 1A | | |
| 1876198 | MGI:432741 | Slc25a20² | Dif-1 carnitine/acylcarnitine | 1.52 | 0.0913 |
| | | | translocase | | |
| 1428627 | MGI:99781 | Smcx | Smith-McCort dysplasia | 1.61 | 0.0053 |
| | | | transcription factor | | |
| 2075523 | MGI:2388097 | Taf3 | TAFII140, RNA polymerase | 0.54 | 0.0001 |
| | | | II TFIID subunit | | |
| 22477947 | MGI:98663 | Tef | Thyrotroph embryonic factor, | 3.12 | 0.0057 |
| | | | transcript variant 1 | | |
| 2247801 | MGI:506974 | Tenc1 | Tensin: C1 containing | 1.53 | 0.0837 |
| | | | phophatase and tensin-like) | | |
| 1768756 | MGI:98767 | Tlm | Tlm oncogene | 0.33 | <0.0001 |
| 1896322 | MGI:450518 | Tra1 | ERp99 (tumor rejection antigen) | 0.63 | 0.014 |
| 1749050 | MGI:106657 | Trim21 | Tripartite motif protein 21 (RNP | 1.65 | 0.001 |
| | | | antigen) | | |
| 2248622 | MGI:507261 | Usp14² | Ubiquitin specific protease | 0.62 | 0.0082 |
| | | | 14 tRNA Guanine | | |
| | | | transglycosylase | | |
| 23958739 | MGI:1346098 | Whac2 | Wolf-Hirschhorn syndrome | 0.61 | 0.0052 |
| | | | candidate 2 | | |
| 2196355 | MGI:501400 | Wwp2 | Ubiquitin protein ligase activity | 3.29 | <0.0001 |
| 1497684 | MGI:278384 | Zfp131 | Zinc finger protein 131 | 0.31 | <0.0001 |
| 2076192 | MGI:2153740 | Zfp358 | Zinc finger protein 358 | 1.55 | 0.0382 |
| 1485629 | MGI:274663 | ZFP454 | Gastrula Zinc finger Protein | 0.32 | 0.0001 |
| | | | (homology to other Zpfs) | | |

Regulated by Genotype when fed 100% Calories

| | | | | | |
|---|---|---|---|---|---|
| 2049046 | MGI:460534 | Adk | Adenosine kinase | 8.39 | 0.0003 |
| 1861999 | MGI:421303 | Apoc1 | Apolipoprotein C1 | 0.19 | 0.0208 |
| 5295934 | MGI:107184 | Cct7 | Chaperonin containing | 5.20 | 0.0024 |
| | | | TCP-1 eta subunit, | | |
| 2049133 | MGI:1298389 | Clecsf8 | C-type (calcium | 0.08 | 0.0377 |
| | | | dependent, carbohydrate- | | |
| | | | recognition domain) lectin | | |
| 1644059 | MGI:333816 | CLMP | Car-Like membrane protein. | 4.84 | 0.0022 |
| | | | CAR = Nrli3 | | |
| 20135640 | MGI:2385923 | Clp1 | Cardiac lineage protein 1 | 0.11 | 0.0143 |
| 2235239 | MGI:532418 | Dbn1 | Debrin like, Abp1 | 5.41 | 0.0003 |
| 1671585 | MGI:1306823 | Dhx | ATP-dependent RNA helicase | 4.30 | 0.0184 |
| | | | (several helicases in mouse | | |
| | | | genome) | | |
| 2041462 | MGI:475249 | Dusp14 | Dual specificity phosphatase 14 | 0.16 | 0.0051 |
| 1528325 | MGI:288619 | Dvl2 | Dishelved 2 | 0.20 | 0.0695 |
| 1380803 | MGI:241712 | E130307J07 | Phox-like and SH3 | 0.18 | 0.0001 |
| 21619380 | MGI:1920992 | EPLIn | Epithelial protein lost | 0.10 | <0.0001 |
| | | | in neoplasm beta | | |
| 1843290 | MGI:2151483 | Flana | Carcinoma related potein, | 4.58 | 0.0107 |
| | | | predicted membrane protein | | |
| 25058755 | MGI:95556 | Flna | Filamin, □ endothelial actin | 8.16 | 0.0988 |
| | | | binding protein | | |
| 1385542 | MGI:242684 | Gnb1l | Guanine nucleotide binding | 5.15 | 0.0459 |
| | | | protein G, β1 | | |
| 2049398 | MGI:458722 | Gosr1 | Golgi SNAP receptor complex | 0.15 | 0.0581 |
| 1853353 | MGI:409816 | Grb2 | Growth factor receptor bound | 11.80 | 0.0478 |
| | | | protein | | |
| 2263030 | MGI:527259 | Gtf2h1 | Basic TF 62 kd protein | 3.89 | 0.0844 |
| 27447549 | MGI:2153839 | Hps3 | Hermansky-Pudlan syndrome, | 9.95 | <0.0001 |
| | | | lysome-related organelle | | |
| | | | complex 3 | | |
| 2307879 | MGI:1333754 | Hrb | HIV Rev binding protein for | 3.80 | 0.0985 |
| | | | nuclear receptor subfamily 1 | | |
| 2041525 | MGI:475131 | Impa2 | Myo-inositol 1(or 4) | 0.11 | 0.0353 |
| | | | monophosphatase | | |
| 1436260 | MGI:260239 | Lad1 | Ladinin | 0.18 | 0.0595 |
| 4395036 | MGI:2153089 | Mrps2 | MRP-S2 mitochondrial | 14.86 | 0.0053 |
| | | | ribosomal protein S2 | | |
| 1529871 | MGI:286942 | Mscp | Mitochondrial solute carrier | 5.17 | 0.0222 |
| | | | protein | | |
| 1934380 | MGI:457875 | Ndufa5 | NADH-ubiquinoone | 4.98 | 0.0172 |
| | | | oxidoreducatase 1□ | | |
| | | | subcomplex 5 | | |
| 2042025 | MGI:344221 | Nssr | Neural-salient serine/ | 5.18 | 0.0827 |
| | | | arginine rich | | |
| 2041988 | MGI:344017 | Oazin | Ornithine decarboxylase | 4.84 | 0.078 |
| | | | antizyme inhibitor | | |
| 13172239 | MGI:108202 | Pcb2 | Poly rC binding 2 | 4.12 | 0.0331 |
| 6942206 | MGI:1342774 | PPargc1 | PPAR□ cofactor 2 | 4.16 | 0.0457 |
| 1654232 | MGI:109494 | Ptprl | Protein tyrosine receptor - □ | 0.19 | 0.0023 |
| 1827407 | MGI:99425 | Rab11b | Rab11b, member RAS oncogene | 0.09 | 0.0217 |
| 1677793 | MGI:325030 | Rcn2 | Reticulocalbin 2 | 5.69 | 0.002 |
| 1684567 | MGI:349892 | Rps6 | Ribosomal protein S6 | 4.33 | 0.0441 |
| 1852997 | MGI:406853 | Ry1 | Ry-1 - putative RNA binding | 0.11 | 0.0176 |
| | | | protein | | |
| 1889593 | MGI:445421 | Slco2b1 | Solute carrier organic anion | 6.58 | 0.0487 |
| | | | transporter 2b1; Slc21a9 | | |
| 1644037 | MGI:334573 | Snf1lk | SNf1-related Kinase | 0.08 | 0.017 |
| 2049149 | MGI:460714 | Tbp | TFIID; TATA binding protein | 0.07 | <0.0001 |
| 1676652 | MGI:98553 | Tcr1 | T-cell receptor alpha chain | 8.79 | 0.0735 |
| 33392729 | MGI:1270128 | Usp12 | Ubiquitin specific protease 1 | 3.91 | 0.0833 |

Regulated by Genotype when fed 70% Calories

| | | | | | |
|---|---|---|---|---|---|
| 1918428 | MGI:469298 | Aldo3 | Fructose-bishosphate aldolase | 0.11 | <0.0001 |
| | | | (MGI87994) | | |
| 1542473 | MGI:253976 | Atf1 | Activating transcription factor | 0.15 | 0.0446 |
| | | | 1 - MHC class II transactivation | | |
| 1918266 | MGI:1201780 | Atp6a1 | Xq terminal portion; lysosomal | 0.09 | 0.0043 |
| | | | accessory protein ATPase | | |
| 1905747 | MGI:456673 | Cars | Cysteinyl-tRNA synthase | 0.09 | 0.0001 |
| 1529944 | MGI:286788 | Ccrl1 | Chemokine (C—C) receptor | 0.08 | 0.0232 |
| | | | like 1 | | |
| 2049207 | MGI:460740 | CD14 | Monocyte/granulocyte cell | 0.13 | 0.0833 |
| | | | surface glycoprotein | | |
| 1672354 | MGI:325973 | Cdc212 | Cyclin dependent kinase | 6.46 | 0.019 |
| 1840745 | MGI:430298 | Chd1l | Chromodomain helicase DNA | 0.09 | <0.0001 |
| | | | binding protein | | |
| 1676738 | MGI:349951 | Cln3 | Ceroid lipofuscinosis, | 0.05 | 0.0104 |
| | | | (MGI:107537) mito membrane | | |
| | | | protein, chaperone/folding | | |
| 1387144 | MGI:243583 | D6Erdt32e | D6Erdt32e- C2 domain = | 0.18 | 0.0017 |
| | | | calcium dependent membrane- | | |
| | | | targeting module | | |
| 1465081 | MGI:266280 | F11r | F11 receptor - Jcam | 0.06 | <0.0001 |
| 1699956 | MGI:354790 | Fn3k | Fructosamine 3 kinase | 15.40 | 0.0346 |
| 1554726 | MGI:293099 | Gdi1 | GDP dissociation inhibitor | 0.06 | 0.0253 |
| 2049398 | MGI:458722 | Gosr1 | Golgi SNAP receptor complex | 0.10 | 0.0124 |
| 1862981 | MGI:427587 | Gpt1 | Glutamic pyruvic | 0.10 | 0.0013 |
| | | | aminotransferase 1 (MGI:95802) | | |
| 1474909 | MGI:267169 | Gsc | Homeobox protein goosecold | 0.10 | 0.0001 |
| 1446915 | MGI:262498 | Hemgn | Hemogen | 0.12 | 0.0569 |
| 1672875 | MGI:324082 | Mtpn | Myotrophin | 0.16 | 0.0697 |
| 2040121 | MGI:468181 | Myadm | Myeloid associated | 0.16 | 0.0912 |
| | | | differentiation factor | | |
| 1476105 | MGI:267803 | Nek6 | Protein-Ser/Thr kinase | 0.09 | 0.0595 |
| 1474962 | MGI:268761 | Nme2 | Nucleoside diphosphate kinase b | 0.11 | 0.0688 |
| 1937333 | MGI:458435 | Oasl1 | 2′-5′ oligoadenylate synthetase- | 0.16 | 0.0716 |
| | | | like 1 | | |
| 1540342 | MGI:2146027 | Pippin | RNA binding protein | 0.12 | 0.0002 |
| 2041865 | MGI:344015 | Prkwnk1 | Protein kinase WNK1 | 6.14 | 0.064 |
| 18249848 | MGI:405041 | Prpf | PRP31 U4/U6 snRNP-associated | 9.19 | 0.0036 |
| | | | 61 kDa | | |
| 2284221 | MGI:532451 | Rab11b | Rab11b | 12.1 | 0.0002 |
| 1355494 | MGI:229033 | Sart3 | Squamous cell carcinoma Ag | 9.35 | 0.0003 |
| | | | recognized by T cells | | |
| 1699511 | MGI:354907 | Sec13r | Secretory protein 13p | 17.94 | 0.0485 |
| 1853537 | MGI:412660 | Smt3ip1 | Sentrin/SUMO specific protease | 0.15 | <0.0001 |
| 1554874 | MGI:894310 | Sept8 | Septin | 0.15 | 0.0386 |
| 1649568 | MGI:334877 | Snx12 | Sorting nexin; SDP8 | 0.06 | 0.0757 |
| 1355309 | MGI:228868 | Spag7 | Single stranded nucleic acid | 0.10 | 0.0118 |
| | | | binding R3h | | |
| 1853387 | MGI:410117 | Stard5 | StAR-related lipid transfer | 0.12 | 0.0365 |
| 1908757 | MGI:453323 | Tal1 | T cell acute lymphocytic | 14.69 | 0.0001 |
| | | | leukemia, transcription factor | | |
| 2235123 | MGI:532383 | Tm4sf2 | PE31/TALLA; tetraspanin 2 | 0.03 | 0.007 |
| 1557815 | MGI:293976 | Trim25 | Tripartite motif, estrogen | 0.07 | 0.0674 |
| | | | responsive finger | | |
| 2187159 | MGI:503077 | Vegfa | Vascular endothelial growth | 0.15 | 0.020 |
| | | | factor | | |

Genotype Regulated Genes

| | | | | | | | |
|---|---|---|---|---|---|---|---|
| 1368954 | MGI:237544 | Aes² | Amino-terminal enhancer | 0.11 | <0.0001 | 0.19 | 0.0344 |
| | | | of split | | | | |
| 1919164 | MGI:469339 | BC022765 | BC022765 (mapped) | 0.11 | 0.0303 | 0.11 | 0.0107 |
| 19343805 | MGI:1098748 | Ctdsp2 | Carboxy-terminal domain, RNA | 0.12 | <0.0001 | 0.13 | 0.0574 |
| | | | Poly II, A) small phosphatase | | | | |
| 1888072 | MGI:406837 | D15Wsu75e | D15Wsu75e (mapped) | 7.36 | 0.0003 | 7.31 | 0.0749 |
| 1904767 | MGI:450848 | Dpysl5 | Collapsin response mediator | 11.31 | 0.0044 | 14.96 | 0.0004 |
| | | | protein 5 (Crmp5); | | | | |
| | | | dihyropyrmidase-like 5 | | | | |
| 1752282 | MGI:375830 | Hcph | Ptp1C phosphatase | 3.97 | 0.0622 | 7.63 | 0.0235 |
| 3098286 | MGI:1342771 | Klf1 | Erythroid kruppel-like factor 1 | 0.16 | 0.0002 | 0.17 | 0.0068 |
| 1332865 | MGI:227638 | Mark4 | MARKL1: MAP/microtubule | 0.18 | 0.03 | 0.14 | 0.0001 |
| | | | affinity-regulating kinase like 1 | | | | |
| 1464182 | MGI:263664 | Tcte1 | T-complex associated testes | 0.12 | <0.0001 | 0.13 | 0.0731 |
| 37590248 | MGI:109637 | Erf | Ets2 repressor factor | 0.19 | 0.0022 | 0.19 | 0.0517 |

Genotype x Diet-Regulated Genes

| | | | | | | | | | | | |
|---|---|---|---|---|---|---|---|---|---|---|---|
| 1769161 | MGI:386336 | Bing4 | TRNA aminoacylation (Bing4) | 5.52 | 0.0013 | | | | | 0.10 | 0.0582 |
| 1905747 | MGI:456673 | Cars | Cysteinyl-tRNA synthase | 11.17 | <0.0001 | | | | | 0.09 | 0.0001 |
| 1711851 | MGI:358020 | Cdc20 | Cdc20 | 2.92 | 0.0234 | | | | | 0.16 | 0.0267 |
| 19484060 | MGI:2135610 | Dnclic1 | Dynein, cytoplasmic, light | 13.15 | 0.0214 | | | | | 0.07 | 0.0168 |
| | | | intermediate chain 1 | | | | | | | | |
| 1464871 | MGI:95557 | Flnc | Filamin, γ | 3.49 | 0.0003 | | | | | 0.16 | 0.0145 |
| 1528898 | MGI:289919 | Man2b | Lysosomal α-mannosidase | 5.84 | 0.0702 | | | | | 0.05 | 0.0785 |
| 1474933 | MGI:266907 | Mrps30 | Mitochondrial ribosomal protein | 3.77 | 0.0001 | | | | | 0.06 | 0.0791 |
| | | | S30 | | | | | | | | |
| 1285745 | MGI:208211 | Nab2 | Ngfl-A binding protein 2 | 3.98 | 0.0001 | | | | | 0.13 | 0.0574 |
| 1446725 | MGI:261583 | Osr1 | Odd skipped | 2.78 | 0.0473 | | | | | 0.10 | 0.0053 |
| 2192713 | MGI:486984 | Pdgfra | PDGF-α receptor | 3.23 | 0.0019 | | | | | 0.15 | 0.0801 |
| 1826093 | MGI:398805 | Rara | Retinoic acid receptor α | 2.84 | 0.0326 | | | | | 0.09 | 0.012 |
| 17389238 | MGI:107484 | Rgl1 | Ral guanine nucleotide | 9.08 | 0.076 | | | | | 0.09 | 0.0989 |
| | | | dissociation stimulator-like | | | | | | | | |
| 2233436 | MGI:517721 | SelM | Selenoprotein SelM | 6.91 | 0.0013 | | | | | 0.14 | 0.0002 |
| 1555534 | MGI:294209 | Shrm | APXL: Actin binding? | 5.84 | 0.0702 | | | | | 0.09 | 0.0269 |
| 1372610 | MGI:237964 | Smo | Smoothened homolog | 5.15 | <0.0001 | | | | | 0.07 | 0.0764 |
| 1333229 | MGI:227262 | Ung | Uracil DNA glycosylase 1 | 4.37 | <0.0001 | | | | | 0.11 | 0.019 |
| 1446336 | MGI:260958 | Mad2l1 | Mitotic arrest deficient | 0.11 | 0.0012 | | | 0.043 | <0.0001 | | |
| 5103143 | MGI:1346040 | Mpdu1 | Mannose-P-dolichol utilization | | | 0.55 | <0.0001 | 4.44 | 0.0055 | | |
| | | | defect | | | | | | | | |
| 1794607 | MGI:403155 | Tere1 | Transepithelia response protein | | | 5.25 | 0.0188 | | | 14.97 | 0.0001 |

TABLE 2
Diet, Genotype, and Diet x Genotype Regulated Genes at “Diabesity” QTL

| QTL¹ | | | | Gene | | | | Gene - Expression Ratio² | | | | Diabetes | |
|---|---|---|---|---|---|---|---|---|---|---|---|---|---|
| Chr | cM | QTL | MGI_ID | Locus | cM | Name³ | MGI_ID | Aal:Acr | Yal:Ycr | Aal:Yal | Acr:Ycr | Association | Ref |
| 1 | 21 | Dbsty1 | 2149843 | ldh1 | 29.8 | Isocitrate | 96413 | 4.68 | | | | Enzyme activity not changed in | [2] |
| | | BW, PG, PI | | | | dehydrogenase 1 (NADP+) | | (<0.0001) | | | | 32 patients with diabetes | |
| | 36⁴ | Insq2 | 1932506 | | | | | | | | | relative to controls | |
| | | SHI | | | | | | | | | | | |
| 2 | 24.5 | Nidd5 | 2154986 | Nek6 | 28 | Never in mitosis-related | 1339708 | | | | 0.09 | β cell mass decreased in Type | [82], |
| | | BW, DI | | | | expressed kinase 6 | | | | | (0.0595) | 2 diabetes, Nek6 regulates | [84] |
| | | | | | | | | | | | | initiation of mitosis | |
| 3 | 2.6 | Insq3 | 1932507 | Hps3 | 12.5 | Hermansky-Pudlak syndrome 3 | 2153839 | | | 99.5 | | HSPs involved in lysosomal | [18]; [86] |
| | | II | | | | | | | | (<0.0001) | | production; Dysfunction of islet | |
| | | | | | | | | | | | | lysosomal system impairs | |
| | | | | | | | | | | | | glucose stimulated release | |
| 5 | 45 | Dbsty2 | 2149844 | Pdgfra | 42 | PDGF-α receptor | 486984 | | | 0.15 | | Interacts with hedgehog, | [99] |
| | | IA | | | | | | | | (0.027) | | involved in insulin production in | |
| | | | | | | | | | | | | pancreas | |
| | 58 | Igfbp3q1 | 1890481 | Shrm | 52 | Shroom | 237964 | 5.84 | | | 0.09 | Regulates cytoarchitecture, | [33]; [47] |
| | | | | | | | | (0.0702) | | | (0.0017) | adherens, and actin dynamics | |
| | | | | | | | | | | | | affect glucose uptake | |
| 6 | 16 | Fglu | 2149370 | Smo | 7.2 | Smoothened homolog | 108075 | 5.15 | | | 0.072 | Receptor for Indian Hedgehog | [45], [99] |
| | | IFG | | | | | | (<0.0001) | | | (0.0017) | (IHH), involved in insulin | |
| | | | | | | | | | | | | signaling in pancreas. Smo | |
| | | | | | | | | | | | | expressed in liver | |
| | | | | Flnc | 8.5 | Filamin | 95557 | 3.49 | | | 0.16 | Insulin causes changes in | [31] |
| | | | | | | | | (0.0003) | | | (0.0145) | cytoskeleton, flilamin A may | |
| | | | | | | | | | | | | interact with insulin receptor | |
| | 35.5⁵ | Nidd3n | 1355301 | Mad2l1 | 30.3 | Mitotic arrest deficient | 186037 | 0.11 | | 0.42 | | Component of mitotic spindle | [70] |
| | | | | | | (homology) - like | | (0.0013) | | (<0.0001) | | assembly checkpoint. May bind | |
| | | | | | | | | | | | | insulin receptor | |
| | | | | Erato | 35.5 | Erato Doi 32 | 243583 | | | 0.18 | 0.18 | C2 domain - Calcium lipid | Eg., [55] |
| | | | | Doie32 | | | | | | (0.0014) | (0.049) | binding domain | |
| | | | | | | | | | | | | IPR000008. Calcium is | |
| | | | | | | | | | | | | important for insulin release | |
| | | | | | | | | | | | | (and other functions | |
| 10 | 46⁶ | Igftbp3q2 | 1890485 | Aes⁹ | 43 | Amino terminal enhancer of split | 88257 | | | 0.11 | 0.19 | Co-repressor of NFκβ,. NFκβ is | [96], [92] |
| | | | | | | | | | | (<0.0001) | (0.012) | activated in insulin resistant | |
| | | | | | | | | | | | | tissues | |
| | | | | Tra1 | 49 | Tumor rejection antigen | 700006 | | 0.63 | | | Chaperonin regulated by | [34] |
| | | | | | | | | | (0.014) | | | glucose, involved in innate and | |
| | | | | | | | | | | | | specific immunity, c | |
| | 59⁶ | Insq4 | 1932516 | Mdm2 | 66 | Double minute 2 | 358044 | 3.36 | | | | May protect B-cell from fatty | [88, 115] |
| | | | | | | | | (0.0009) | | | | acid induced apoptosis; | |
| | | | | | | | | | | | | interacts with P53, involved in | |
| | | | | | | | | | | | | insulin receptor 3 regulation | |
| 11 | 2 | Nidd4n | 1355273 | Dbn1 | 1 | Debrin | 97919 | | | 5.31 | | Debrin = mABP1 = SH3P7, a | [46], [23] |
| | | | | | | | | | | (0.0003) | | target of Scr tyrosine kinase. | |
| | | | | Dvl2 | 3.5 | Disheveled 2 | 98553 | | | 0.19 | | Implicated in cytoskeletal | |
| | | | | | | | | | | (0.0695) | | regulation, endocytosis, cAMP | |
| | | | | | | | | | | | | signaling | |
| | | | | | | | | | | | | Involved in complexes | [85], |
| | | | | | | | | | | | | regulating Wnt pathyway. Wnt | |
| | | | | | | | | | | | | inhibits glycogen synthase | |
| | | | | | | | | | | | | kinase-3 stabilizing β-catenin. | |
| | | | | | | | | | | | | GSK3 involved in T2DM? | |
| | 31 | Nidd4n | 1355320 | Nhp2 | 28.5 | Nucleolar protein family | 545881 | | 1.98 | | | Indirect? - Member of a | [32] |
| | | | | | | A Member 2 | | | (<0.0001) | | | complex involved in nucleolar | |
| | | | | | | | | | | | | RNA processing | |
| | | | | Sept8 | 28.75 | Septin 8 | 894310 | | | | 0.15 | Cell division and chromosome | |
| | | | | | | | | | | | (0.0386) | partitioning | |
| | | | | | | | | | | | | No direct studies | |
| | | | | Skpa1 | 31 | S-phase kinase-associated | 479801 | | 0.6 | | | In Arabidopsis, interacts with | [97] |
| | | | | | | protein | | | (0.0015) | | | AtGRH1, a homolog of yeast | |
| | | | | | | | | | | | | GRR1, involved in glucose | |
| | | | | | | | | | | | | repression. | |
| | | | | Mpdu1 | 39 | Mannose-P-Dolichol | 497018 | | 0.55 | 4.44 | | Helix-loop-helix transcription | [64], [4] |
| | | | | | | utilization defect | | | (<0.0001) | (0.0055) | | factor involved in pancreatic | |
| | | | | | | | | | | | | development and muscle | |
| | | | | | | | | | | | | function | |
| | 69 | Nidd4 | 68 | Fasn | 72 | Fatty acid synthase | 351529 | | 1.98 | | | Expression regulated by insulin | [95], [19] |
| | | | | | | | | | (<0.0001) | | | and glucose | |
| | | | | Grb2 | 75 | Growth factor receptor | 409816 | | | 11.8 | | Increased association of IRS- | [98] |
| | | | | | | bound protein 2 | | | | (0.0478) | | 1/phosphatidylinositol 3-kinase, | |
| | | | | | | | | | | | | IRS-1/growth factor receptor | |
| | | | | | | | | | | | | bound 2 (Grb2), and Shc/Grb2 | |
| | | | | | | | | | | | | in diabetic rats | |
| 12 | 48 | Dbsty3 | 2149845 | Gsc | 52 | Homeobox protein | 107717 | | | | 0.10 | None - Gsc involved in | |
| | | BMI, IA, | | | | goosecoid | | | | | (0.079) | development | |
| | | LL | | | | | | | | | | | |
| 14 | 22.5 | Nidd2n | 1355273 | Tcr1 | 19.5 | T-cell receptor alpha | 98553 | | | 8.79 | | No information, immune | |
| | | IGT | | | | | | | | ().0735) | | functions important in diabetes | |
| 15 | 49.6 | Dbsty4 | 2149846 | Gpt1 | 40.3 | Glutamic pyruvic | 95802 | | | | 7.31 | Diabetics are magnesium | [14, 30, 59] |
| | | II | | | | ttransaminase | | | | | (0.731) | deficient; alterations in signal | |
| | | | | | | | | | | | | transduction? Cleaves PO_{4 f}rom | |
| | | | | | | | | | | | | TAK1, involved in inflammation | |
| | | | | D15Wsu | 46.7 | Uncharacterized | 106313 | | | 7.36 | 7.31 | | |
| | | | | 75e | | | | | | (0.0003) | (0.731) | | |
| | | | | Tef | 46.7 | Thyrotroph embryonic | 523917 | | 3.12 | | | In thymus, involved in calcium | [51] |
| | | | | | | factor, transcript variant | | | (0.0057) | | | responsive genes expression | |
| | | | | H1fo | 46.75 | Histone H1 | 523960 | | 0.33 | | | No information - H1 involved in | |
| | | | | | | | | | (0.0001) | | | chromatin structure | |
| 17 | 56.7⁶ | Insq5 | 1932517 | Ppmb1⁹ | 50.8 | Protein phosphatase 1B, | 523448 | | 0.57 | | | PTP1B as a negative regulator | [79] |
| | | | | | | Mg²⁺, β | | | (0.0001) | | | of insulin action | |
| 18 | 16⁵ | Nidd2 | 1227794 | Fgf1 | 19 | Fibroblast growth factor | 534640 | | 1.59 | | | Involved in insulin secretion in | [78] |
| | | HG | | | | | | | (0.0102) | | | pancreas | |

¹QTL loci description, numbers refer to specific loc
Dbsty diabesity
Insq insulin QTL
Nidd non-insulin-dependent diabetes mellitus
Niddn non-insulin-dependent diabetes mellitus in NSY
T2dm type 2 diabetes mellitus:
Igfbp3 insulin-like growth factor binding protein
BMI = Body Mass Index
BW = Body weight
DI = Decreased insulin
F = Fasting
HG = Hyperglycemia
IA = Increased adiposity
IFG = Increased fasting glucose
I:G = Insulin:glucose ratio
IGT = Impaired glucose tolerance
II = Increased insulin
Insq - Insulin levels
LL = Leptin Levels
PI = Plasma insulin
PG = Plasma glucose
SHI = Susceptibility to hyperinsulinemia
²See Table 1
³Color code:
Red - Signal transduction, kinases, cell cycle, apoptosis
Orange - Transcription
Brown - Splicing genes
Black - Unknown
Purple - Immune function
Blue - Metabolism
Green - Structural including transporters
Light Blue - Protease
⁴⁻⁸Human T2DM QTL (rev. in [21])
⁴2q24.2 Marker: (D2S2345)
⁵4q34.1 (D421539)
⁶12q25 (D12S375)
⁷2p21 (DS2259)
⁸5q21.1 (D5S816)
⁹Differentially expressed in muscle between human with and without family history of DM, Table 3 [74]

TABLE 3
Diet, Genotype, and Genotype X Diet Regulated Genes Mapping to Obesity and Weight Gain QTL

| QTL | | | | Gene | | | | Expression Ratio | | | | | |
|---|---|---|---|---|---|---|---|---|---|---|---|---|---|
| Chr | cM | Name | MGI_# | Locus | cM | Function | MGI_# | Aal:Acr | Yal:Ycr | Aal:Yal | Acr:Ycr | Obesity Association | Ref |
| 1 | 25 | Wt10q1 | 1344340 | Idh1* | 29.8 | Isocitrate dehydrogenase | 523407 | 4.68 | | | | Upregulated in lean/obesity | [9] |
| | 27 | Wt6q1 | 1344348 | | | | | (<0.0001) | | | | resistant perilipin KO male | |
| | 28.7 | Obq | 2150696 | | | | | | | | | mice (no strain of diet info) | |
| | 36 | Bw57 | 131668 | | | | | | | | | | |
| | 88.4 | Obq9 | 2150698 | F11r | 93.3 | F11 receptor, Jcam | 266280 | | | | 0.06 | Increased cell adhesion | [52] |
| | | | | | | | | | | | (<0.0001) | molecules in obese, | |
| | | | | | | | | | | | | hyperlipidemic patients | |
| | | | | Pea15 | 93.8 | Phosphoprotein enriched in | 1097689 | 1.52 | | | | Increases glucose uptake, | [12]; [113] |
| | | | | | | diabetes, death effector | | (0.0909) | | | | impairs insulin action; | |
| | | | | | | domain | | | | | | SNPs in gene not | |
| | | | | | | | | | | | | associated with 50 T2DM in | |
| | | | | | | | | | | | | Pima Indians | |
| 3 | 49 | Bglq3 | 108562 | Ampd2 | 50.4 | AMP deaminase | 522039 | | 0.63 | | | Adenosine increases | [29] |
| | 61 | Wt10q2 | 1344339 | | | | | | (0.0066) | | | responsiveness of muscle | |
| | | | | | | | | | | | | glucose transport to insulin | |
| | | | | Fabp2 | 55 | Fatty acid binding protein, | 419365 | 13.85 | | | | A54T SNP linked to insulin | [108] |
| | | | | | | intestinal (adipocyte) | | (0.0497) | | | | sensitivity in obese or with | |
| | | | | | | | | | | | | high fat diet | |
| 4 | 55 | Bwtq2 | 1891194 | Cyp4a10 | 49.5 | Cytochrome P450 Cyp4a | 345223 | | 0.62 | | | Similar to CYP4A11, a fatty | [65] |
| | 59 | Bw7 | 1316684 | | | | | | ().0084) | | | acid omega hydroxylase. | |
| | | | | | | | | | | | | Increased FAs linked to | |
| | | | | | | | | | | | | insulin resistance | |
| | | | | Tal1 | 49.5 | T cell acute lymphocytic | 453323 | | | | 14.69 | | |
| | | | | | | leukemia | | | | | (0.0001) | | |
| 5 | 42 | Bw8 | 1316643 | Pdgrfra* | 42 | Platelet derived growth | 486984 | 3.23 | | | 0.15 | Interacts with hedgehog, | [99] |
| | 44 | Bwob | 2149826 | | | factor □receptor | | (0.0019) | | | (0.0801) | involved in insulin | |
| | | | | | | | | | | | | production in pancreas | |
| | 81 | Bw13 | 1889214 | Mdh2 | 78 | Malate dehydrogenase | 442591 | | 1.59 | | | Increased expression in | [10] |
| | | | | | | | | | (0.0106) | | | obese high fat fed mice | |
| | | | | | | | | | | | | relative to lean | |
| 6 | 3.05 | Mob2 | 99506 | D6Wsu176e | 2 | Predicted osteoclast protein | 505835 | | 0.63 | | | | |
| | 4 | Bwtq3 | 1891195 | | | | | | (0.0271) | | | | |
| | 16 | Fglu | 2149370 | Smo* | 7.2 | Smoothened homolog | 237964 | 5.15 | | | 0.07 | Receptor for Indian | [45], [99] |
| | | | | | | | | (<0.0001) | | | (0.0794) | Hedgehog (IHH), involved | |
| | | | | | | | | | | | | in insulin signaling in | |
| | | | | | | | | | | | | pancreas. Smo expressed | |
| | | | | | | | | | | | | in liver | |
| | | | | Flnc | 8.5 | Filamin | 95557 | 3.49 | | | 0.16 | Insulin causes changes in | [31] |
| | | | | | | | | (0.0003) | | | (0.0145) | cytockeleton, flilamin A may | |
| | | | | | | | | | | | | interact with insulin | |
| | | | | | | | | | | | | receptor | |
| | 26.8 | Obq13 | 2150702 | Mad2l1* | 30.3 | Mitotic arrest deficient | 260958 | 0.11 | | 0.43 | | | [77] |
| | 35 | Bw18 | 1932503 | | | | | (0.0012) | | (<0.0001) | | | |
| | 43.5 | Obq14 | 2150703 | D6Erato Doi | 35.5 | Ca²⁺ dependent membrane targeting | 243583 | | | | 0.18 | Low dietary Ca²⁺ linked with | [117] |
| | | | | 32 | | | | | | | (0.0017) | obesity | |
| 8 | 45 | Wg3 | 1933814 | Es22 | 43.2 | Liver carboxylesterase 22 | 421248 | 15.61 | | | | Xenobiotic metabolism, | [87] |
| | | | | | | (EC 3.1.1.1) | | (0.0173) | | | | short and acyl glycerols, | |
| | | | | | | | | | | | | acyl-CoA, Vitamin A esters, | |
| | | | | | | | | | | | | and acyl-carnitine | |
| | | | | | | | | | | | | hydrolyzing activities in | |
| | | | | | | | | | | | | vitro, | |
| | 56 | Bwq3 | 1890413 | Cdh3 | 53.3 | OL-protocadherin | 88356 | 2.97 | | | | Maintains cell cell | [91] |
| | | | | | | | | (0.0703) | | | | interactions in pancreas | |
| | | | | | | | | | | | | (and other) | |
| 9 | 8 | Bwtq4 | 2150123 | Ets1 | 15 | Ets 1 - transcription | 442687 | | 2.45 | 0.36 | 0.31 | | |
| | 15 | Obq5 | 2349407 | | | | | | (<0.0001) | | | | |
| | 60 | Dob2 | 99950 | Cacna2d2 | 60 | Ca²⁺ channel protein □-2 | 99916 | 3.19 | | | 0.38 | Required for intracellular | [8] |
| | | | | | | | | (0.0135) | | | | signaling and mitochondrial | |
| | | | | | | | | | | | | membrane integrity | |
| 11 | 27.8 | Bwtq5 | 2150125 | Nhp2* | 28.5 | Nucleolar protein family A | 545881 | | 1.98 | | | Site-specific | [75] |
| | 32 | Wt10q3 | 1344409 | | | | | | (0.0001) | | | pseudouridylation of rRNAs, | |
| | 36 | Wt6q3 | 1344346 | | | | | | | | | a component of telomerase | |
| | | | | Sept8* | 29.75 | Septin 8 | 1194505 | | | | 0.15 | Guanine nucleotide binding | [48] |
| | | | | | | | | | | | (0.0386) | protein involved in | |
| | | | | | | | | | | | | cytokinesis | |
| | | | | Skpa1* | 29.75 | S phase kinase associated | 479801 | | 0.9 | | | A component of SCF | [68], [39] |
| | | | | | | protein A1 | | | (0.0015) | | | ubiquitin ligases, link cell | |
| | | | | | | | | | | | | and centrosome cycles; in | |
| | | | | | | | | | | | | yeast, regulated by glucose | |
| | | | | Mpdu1* | 39 | Mannose-P-dolichol | 1346040 | | 0.55 | 4.44 | | Involved in glycosylation | [50] |
| | | | | | | utilization defect | | | (<0.0001) | (0.005) | | | |
| 11 | 46 | Wg4 | 1933816 | Spag7 | 42 | Single stranded nucleic acid | 228868 | | | | 0.10 | Similar to adipocyte- | [49] |
| | 55 | Bw4 | 316681 | | | binding R3h | | | | | (0.112) | specific serum protein, | |
| | | | | | | | | | | | | Acrp-30, similar to C1q, but | |
| | | | | | | | | | | | | unknown function | |
| | | | | Flana | 42 | Carcinoma related protein - | 2151483 | | | | 4.58 | None | |
| | | | | | | membrane | | | | | (0.0107) | | |
| | | | | Dusp1 | 48 | Dual specificity | 475249 | | | | 0.16 | No direct: Dusp is a | [105], [66] |
| | | | | | | phosphatase 14 (similar) | | | | | (0.0051) | negative regulator of CD28, | |
| | | | | | | | | | | | | which in turn regulates | |
| | | | | | | | | | | | | insulin-like growth factor-I | |
| | | | | | | | | | | | | receptor | |
| | | | | Sfrs1 | 49 | Splicing factor, arg/ser-rich | 98283 | 0.29 | | | | No direct link | |
| | | | | | | 1 (ASF/SF2) | | (0.0058) | | | | | |
| | | | | Rara | 57 | Retinoic acid receptor α | 3988-5 | 2.84 | | | 0.09 | Inverse relationship | [80] |
| | | | | | | | | (0.0326) | | | (0.012) | between PPAR□ and | |
| | | | | | | | | | | | | RAR□ expressions in | |
| | | | | | | | | | | | | human adipose tissue in | |
| | | | | | | | | | | | | obese individuals | |
| 12 | 53 | Mob3 | 105951 | Gsc* | 52 | Homeobox Gooseocoid | 267169 | | | | 0.1 | Transcription factor in | |
| | | | | | | | | | | | (0.0001) | development | |
| 13 | 10 | Bw15 | 1889216 | Nid1 | 7 | Nidogen/Entactin | 431735 | | 2.86 | | | Component of basement | |
| | | | | | | | | | (<0.0001) | | | membrane zones | |
| | 6 | Bw11 | 1316645 | Npr3 | 6.7 | Natriuretic peptide receptor 3 | 97763 | | 0.62 | | 4.77 | NP are powerful lipolytic | [90] |
| | 6.7 | Mob4 | 105950 | | | | | | (0.0117) | | | agents in subcutaneous fat | |
| | 12.3 | Dob9 | 1201669 | | | | | | | | | cells | |
| 15 | 41.2 | Bwtq6 | 2150126 | Gpt1* | 40.3 | Glutamic pyruvic | 95802 | | | | 7.31 | Diabetics are magnesium | [14, 30, 59] |
| | | | | | | transaminase | | | | | (0.731) | deficient; alterations in | |
| | | | | | | | | | | | | signal transduction? | |
| | | | | | | | | | | | | Cleaves PO₄_{f}rom TAK1, | |
| | | | | | | | | | | | | involved in inflammation | |
| | | | | D15Wsu75e | 46.7 | Uncharacterized | 106313 | | | 7.36 | 7.31 | | |
| | | | | | | | | | | (0.0003) | (0.731) | | |
| | | | | Tef | 46.7 | Thyrotroph embryonic factor, | 523917 | | 3.12 | | | In thymus, involved in | [51] |
| | | | | | | transcript variant | | | (0.0057) | | | calcium responsive genes | |
| | | | | | | | | | | | | expression | |
| | | | | H1fo* | 46.75 | Histone H1 | 523960 | | 0.33 | | | No information - H1 | |
| | | | | | | | | | (0.0001) | | | involved in chromatin | |
| | | | | | | | | | | | | structure | |
| 17 | 4 | Obq4 | 1100507 | Tbp | 8.254 | TFIID, TATA binding protein | 460714 | | | 0.07 | | Activation of insulin gene | [38] |
| | | | | | | | | | | (<0.0001) | | transcription involves TFIID | |
| | | | | | | | | | | | | to the insulin promoter via | |
| | | | | | | | | | | | | its interaction with | |
| | | | | | | | | | | | | hTAF(II)130 and TBP | |
| | 23 | Trbv4c1 | 1927660 | Vegfa | 24.2 | Vascular endothelial growth | 103178 | | | | 0.15 | Regulated in part by | [3, 13, 22, 100] |
| | | | | | | factor-3 | | | | | (0.020) | glucose; High glucose, low | |
| | | | | | | | | | | | | VEGF = microvascular | |
| | | | | | | | | | | | | complications; low glucose, | |
| | | | | | | | | | | | | high VEGF = sporadic | |
| | | | | | | | | | | | | proliferative events? | |
| 18 | 28 | Bwq4 | 1890414 | CD14 | 31 | Monocyte/granulocyte cell surface | 460740 | | | | 0.13 | With □c subunit: STAT5 & | [89] |
| | | | | | | glycoprotein | | | | | (0.833) | JAK2 signaling, glucose | |
| | | | | | | | | | | | | transport, inflammatory | |
| | | | | | | | | | | | | responses | |
| X | 26.4 | Bw19 | 2150137 | Fina | 29.8 | Filamin | 95556 | | | | 8.16 | Interacts with insulin | [31] |
| | 32 | C10bw6 | 2137243 | | | | | | | | (0.099) | receptor, modulates | |
| | | | | | | | | | | | | response | |
| | | | | Atp6a1 | 29.83 | Lysosomal accessory protein | 469414 | | | | 0.09 | Acidificaton of lysosome for | |
| | | | | | | ATPase | | | | | (0.0043) | endocytosis of receptors | |
| | | | | Gdi1 | 29.83 | GDP dissociation inhibitor | 293099 | | | | 0.06 | Signal transduction, | [83] |
| | | | | | | | | | | | (0.0253) | vacuolar functions, sorting | |
| | 59.5 | Dob7 | 1195259 | Jarid1c | 64 | Smith-McCort syndrome transcription | 104566 | | 1.61 | | | negative regulation of cell | [102] |
| | | | | | | factor | | | (0.0053) | | | proliferation signaling. | |

QTL loci description, numbers refer to specific loci
Bglu blood glucose level
Bw body weight
Bwefm body weight females and males day 10
Bwem body weight day 30 males
Bwf body weight and fat
Bwob body weight of obese males
Bwt Body weight
C10bw castaneus 10 week body weight
Dob dietary obesity
Mob multigenic obesity
Mors modifier of obesity related sterility
Obq obesity
Trbv4c T cell receptor beta variable 4, control
Wg weight gain in high growth mice
Wt10 body weight, 10 weeks
Wt3 body weight, 3 weeks
Wt6 body weight, 6 weeks
¹⁰Same as Table 1
¹¹Color code:
Red - Signal transduction, kinases, cell cycle, apoptosis
Orange - Transcription factors
Brown - Splicing
Blue - Metabolisms and enzymes
Purple - Immune function
Green - Structural including transporters
Light Blue - Protease
Black - unknown
*Overlap with diabetes genes (Table 2)

1. A microarray for screening for the presence of variants (alleles) of one or more diet-regulated disease-associated genes, the microarray comprising at least one nucleotide variant selected from the group consisting of: the genes listed in Table 2.

2. A microarray for screening for the presence of variants (alleles) of one or more diet-regulated disease-associated genes, the microarray comprising the genes listed in Table 2.

3. A method for determining, in an individual, an association between an allele of a diet-regulated disease-associated gene and a diet-associated phenotypic response, by performing the following steps: (1) determining which allele(s) are present in the individual, (2) feeding the individual a first diet, (3) determining the phenotype of the individual in response to the first diet, (4) feeding the individual a second diet, (5) determining the new phenotype of the individual and (6) determining an association between an allele of a diet-regulated disease-associated gene and a diet-associated phenotype.
